(19) United States
(12) Patent Application Publication
(10) Pub. No.: US 2022/0414525 A1
(43) Pub. Date: Dec. 29, 2022

(54) MACHINE LEARNING APPROACH FOR SOLVING BEAM ANGLE OPTIMIZATION

(71) Applicant: VARIAN MEDICAL SYSTEMS, INC., Palo Alto, CA (US)

(72) Inventors: Santiago Gaspar PRINC, Helsinki (FI); Perttu NIEMELA, Helsinki (FI); Tuomas JYSKE, Helsinki (FI); Reynald van der STRAETEN, Machelen (BE)

(73) Assignee: VARIAN MEDICAL SYSTEMS, INC., Palo Alto, CA (US)

(21) Appl. No.: 17/356,235

(22) Filed: Jun. 23, 2021

(51) Int. Cl. G06N 20/00; G16H 20/40; G16H 30/40

(52) U.S. Cl. CPC G06N 20/00; G16H 20/40; G16H 30/40

(57) ABSTRACT

Embodiments described herein provide for revising radiation therapy treatment plans, and in particular, revising beam angles used during radiation therapy treatment. A computer may receive a radiation therapy treatment plan based on a particular patient's diagnosis. The computer may use a machine learning model to revise radiation therapy treatment parameters such as a beam angle indicating a direction of radiation into the patient. The machine learning model may use reinforcement learning to optimize an initial beam angle from the radiation therapy treatment plan, revising the beam angle. The performance of the machine learning model is measured against metrics including fulfilling dosimetric clinical goals. The machine learning model may present the revised beam angle for display to a medical professional, or transmit the revised beam angle to downstream applications to further revise the radiation therapy treatment plan.

### TECHNICAL FIELD

This application relates generally to using artificial intelligence modeling to optimize beam angles in radiation therapy treatment procedures.

### BACKGROUND

Radiotherapy (radiation-based therapy) is used as a cancer treatment by emitting high doses of radiation that can kill cells or shrink a tumor. Due to the extreme nature of the radiation emitted from the radiation therapy machine, and in particular, of proton therapy, it is imperative that treatment attributes are precisely calculated and followed. For example, a beam angle defines a direction in which a radiotherapy machine emits radiation particles. The beam angle should be positioned (or set) such that the target region of a patient's anatomy that is intended to receive radiation (e.g., a tumor, referred to as the planning target volume (PTV)) receives enough radiation to kill the cancerous cells during the radiotherapy treatment. However, other organs or anatomical regions that are adjacent to, or surrounding, the PTV can be in the way of radiation beams (directed via the beam angle) and can receive enough radiation to damage or harm such organs or anatomical regions. These organs or anatomical regions are referred to as organs at risk (OARs).

Usually a physician or a radiologist identifies both the PTV and the OARs prior to radiotherapy using, for example, computed tomography (CT) images, cone beam CT images (CBCT), four-dimensional CT images (e.g., CT images over time), magnetic resonance imaging (MRI) images, positron emission tomography (PET) images, ultrasound images, images obtained via some other imaging modality, or a combination thereof. The physician or the radiologist may manually mark the PTV and/or the OARs on the medical images of the patient.

Radiation therapy treatment attributes refer to attributes of how the patient's treatment is implemented. Radiation therapy treatment attributes include attributes of a radiation therapy machine while the patient is receiving the prescribed radiotherapy dose and other radiation parameters (e.g., radiation intensity, beam angle, radiation type, number of beam fields (field geometry)) indicating how the dosage is delivered to the patient's anatomy. Using the medical images of the patient as well as the identified PTV and the OARs, a team of medical professionals (e.g., physicians, radiologists, oncologists, radiology technicians, other medical personnel or a combination thereof) determines the beam angles and other radiation parameters and radiation therapy treatment attributes to be used during the radiotherapy treatment. In determining these and other parameters, the medical professional attempts to set the beam angles in a treatment plan to achieve a radiation dose distribution to deliver to the patient that meets predefined criteria (also referred to herein as the clinical goals). Such clinical goals can include dosimetric metrics (e.g., predefined radiation dose thresholds, ranges for the PTV and the OARs, sensitivities of the PTV and/or OARs), a robustness measure, relative biological effects, and linear energy transfer metrics, among others.

Conventionally, beam angle optimization and other radiation therapy treatment attributes and radiation parameters are identified as part of a treatment plan for particular patients with particular tumors. For various types of tumors, medical professionals have determined, based on past experiences and trial and error, common beam angles and other radiation parameters. However, this conventional method is inefficient because it is error-prone, and relies heavily on the medical professional's subjective understanding and skills. Furthermore, this conventional process is time-consuming and tedious.

### SUMMARY

For the aforementioned reasons, there is a need to optimize the beam angles in a treatment plan to improve the resulting quality of the treatment plan. There is a need for a beam angle optimization system that optimizes beam angles and other radiation parameters given a treatment plan. The task of learning how to adapt beam angles to better suit specific patients is translated from being a medical professional task, into being a task executed using a trainable machine learning model. As discussed herein, the angle of radiation beams administered to a patient is optimized to improve the clinical outcome of the targeted radiation during treatment. Disclosed herein are systems and methods capable of recommending beam angle modifications to optimize the patient's treatment plan in a manner that does not overrule medical professionals. In some implementations, a medical professional may propose an initial treatment plan to be optimized by the systems and methods described herein. In other implementations, systems and methods disclosed herein may not depend on a medical professional's subjective skills and understanding. For example, a processor may utilize the systems and methods described herein to optimize an initial treatment plan (e.g., treatment templates provided for a patient). The beam angles are optimized using an end-to-end machine learning model such that the beam angles can be used for guiding clinical plan optimization and to save calculation time. Rather than optimizing certain aspects of the beam angle or generating possible beam angles using an algorithm, the end-to-end machine learning model holistically optimizes the beam angle by ingesting, in some implementations, a point cloud representation of the structures segmented from medical images, clinical goals, and a treatment plan. In other implementations, an automatic image segmentation module may automatically segment PTVs and/or OARS in medical images. The medical images may be segmented, or otherwise analyzed to identify different contrasts in the medical image. The segmented medical images may subsequently be ingested by the end-to-end machine learning model. In yet other implementations, the end-to-end system may ingest medical images. The machine learning model recommends modifications to a base treatment plan (including beam angles) at a higher response time (once trained), is scalable (e.g., may be implemented using federated learning to update a central policy model), and may be extended (e.g., the core policy model is modular and can be modified with little to no impact beyond retraining). A flexible framework may be applied to many manifestations of the beam angle optimization problem. For example, beam angles may be optimized with respect to proton radiation as well as photon radiation.

The machine learning model replicates (or simulates) the way in which a dosimetrist would gain expertise on how to position the beams, and subsequently uses the trained model to recommend beam angle modifications that are predicted to result in a more performant treatment plan. The machine learning model receives a treatment plan that may include initial beam angles, PTV information, OAR information, medical images and various clinical goals. An environment, such as the anatomical structure of the patient and the positioning of the radiation fields, may be external to the machine learning model. Representations of the environment may be fed into the machine learning model. Agents operating in parallel on each environment, asynchronously select actions for the corresponding environment based on global policies and values. The machine learning model outputs a tensor indicating revised (or optimized, improved, and recommended) modifications to be applied to a beam angle (or a set of beam angles) for display to a user and/or as an input various other downstream applications. The beam angles may already be defined in a base treatment plan but modified based on the output of the machine learning model.

In an embodiment, a computer-implemented method may comprise executing, by at least one processor, a machine learning model that receives an input of data associated with a treatment plan for a patient and outputs a beam angle for the patient indicating a direction of radiation into the patient, wherein the machine learning model is trained using a training dataset comprising a training treatment plan and a corresponding score, wherein the machine learning model iteratively calculates a reward, using a policy, for a possible beam angle for the training treatment plan in the training dataset; and transmitting, by the at least one processor, the beam angle to a second processor.

The treatment plan and the training treatment plan may comprise at least one of a medical image, a clinical goal, a planning target volume, an organ at risk, a radiation type, a radiation dose, an initial beam angle, or a field geometry.

The medical image may include at least a structure of the planning target volume or a structure of the organ at risk.

The computer-implemented method may further comprise executing the machine learning model that receives the input of data associated with the treatment plan for the patient and outputs a field geometry, wherein the machine learning model is trained using a training dataset comprising the training treatment plan and a corresponding score.

The computer-implemented method may further comprise presenting, by the processor, for display, the beam angle.

The machine learning model may be trained using asynchronous advantage actor critic reinforcement learning.

The machine learning model may be implemented using hybrid graphics processing units and central processing units.

The machine learning model may be optimized with respect to one or more clinical goals received in the treatment plan, the clinical goals including at least one of a dosimetric quality, a robustness measure, metrics based on linear energy transfer, or relative biological effects.

The computer-implemented method may further comprise receiving, from the second processor, a revised treatment plan, wherein the revised treatment plan is based on the beam angle; executing, by the at least one processor, the machine learning model using the revised treatment plan for the patient and outputting a revised beam angle; and transmitting, by the at least one processor, the revised beam angle to the second processor.

Iteratively calculating the reward, using the policy, for the possible beam angle from the training treatment plan in the training dataset may include iteratively comparing the reward to a baseline.

In another embodiment, a system may comprise a server comprising a processor and a non-transitory computer-readable medium containing instructions that when executed by the processor causes the processor to perform operations comprising: execute a machine learning model that receives an input of data associated with a treatment plan for a patient and outputs a beam angle for the patient indicating a direction of radiation into the patient, wherein the machine learning model is trained using a training dataset comprising a training treatment plan and a corresponding score, wherein the machine learning model iteratively calculates a reward, using a policy, for a possible beam angle for the training treatment plan in the training dataset; wherein the machine learning model iteratively increases a summation of rewards until the policy satisfies an accuracy threshold; transmit the beam angle to a second processor.

At least one of the treatment plan or the training treatment plan may comprise at least one of a medical image, a clinical goal, a planning target volume, an organ at risk, a radiation type, a radiation dose, an initial beam angle, or a field geometry.

The medical image may include at least a structure of the planning target volume or a structure of the organ at risk.

The processor may be further configured to execute the machine learning model that receives the input of data associated with the treatment plan for the patient and outputs a dose distribution, wherein the machine learning model is trained using a training dataset comprising the training treatment plan and a corresponding score.

The processor may be further configured to present for display, the beam angle.

The machine learning model may be trained using asynchronous advantage actor critic reinforcement learning.

The machine learning model may be implemented using hybrid graphics processing units and central processing units.

The machine learning model may be optimized with respect to one or more clinical goals received in the treatment plan, the clinical goals including at least one of a dosimetric quality, a robustness measure, metrics based on linear energy transfer, or relative biological effects.

The processor may be further configured to receive, from the second processor, a revised treatment plan, wherein the revised treatment plan is based on the beam angle; execute the machine learning model using the revised treatment plan for the patient and outputting a revised beam angle; and transmit the revised beam angle to the second processor.

Iteratively calculating the reward, using the policy, for the possible beam angle from the training treatment plan in the training dataset may include iteratively comparing the reward to a baseline.

### BRIEF DESCRIPTION OF THE DRAWINGS

Non-limiting embodiments of the present disclosure are described by way of example with reference to the accompanying figures, which are schematic and are not intended to be drawn to scale. Unless indicated as representing the background art, the figures represent aspects of the disclosure.

FIG. 1 illustrates components of a beam angle optimization system, according to an embodiment.

FIG. 2 illustrates a flow diagram of a process executed in a beam angle optimization system, according to an embodiment.

FIG. 3A illustrates an example of a simplified reinforcement learning model, according to an embodiment.

FIG. 3B illustrates an example of an asynchronous advantage actor critic reinforcement learning model, according to an embodiment.

FIG. 4 illustrates an example recommendation of beam angles based on a treatment plan, according to an embodiment.

FIG. 5 illustrates a non-limiting visual example of a workflow utilizing the methods and systems described herein, according to an embodiment.

FIG. 6 illustrates another non-limiting visual example of a workflow utilizing the methods and systems described herein, according to an embodiment.

### DETAILED DESCRIPTION

Reference will now be made to the illustrative embodiments depicted in the drawings, and specific language will be used here to describe the same. It will nevertheless be understood that no limitation of the scope of the claims or this disclosure is thereby intended. Alterations and further modifications of the inventive features illustrated herein, and additional applications of the principles of the subject matter illustrated herein, which would occur to one skilled in the relevant art and having possession of this disclosure, are to be considered within the scope of the subject matter disclosed herein. Other embodiments may be used and/or other changes may be made without departing from the spirit or scope of the present disclosure. The illustrative embodiments described in the detailed description are not meant to be limiting of the subject matter presented.

Radiotherapy clinics may utilize software solutions for executing radiation therapy. The software solutions may analyze patient data, clinical goals, and a multitude of other factors to generate a customized radiation treatment plan (e.g., dose distribution, radiation parameters, side effect prediction, organ and/or tumor segmentation, machine therapy attributes, treatment frequency, treatment timing, treatment modalities). The radiation parameters may include an optimized beam angle for a patient and one or more numbers of beams (e.g., field geometry settings). The radiation parameters may be improved (selected, recommended, and/or optimized) based on considering one or more clinical goals such as a dosimetric goodness function, a robustness, biological effects of radiation, among others.

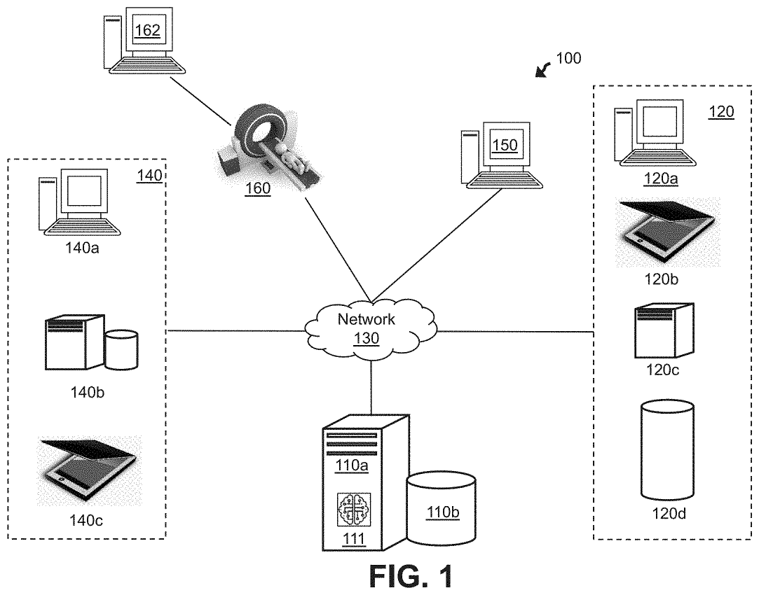

FIG. 1 illustrates components of beam angle optimization system 100, according to an embodiment. The system 100 may include an analytics server 110a, system database 110b, machine learning models 111, electronic data sources 120a-d (collectively electronic data sources 120), end-user devices 140a-c (collectively end-user devices 140), an administrator computing device 150, and a medical device 160 having a medical device computer 162. Various components depicted in FIG. 1 may belong to a radiotherapy clinic at which patients may receive radiotherapy treatment, in some cases via one or more radiotherapy machines located within the clinic (e.g., medical device 160). The above-mentioned components may be connected to each other through a network 130. Examples of the network 130 may include, but are not limited to, private or public LAN, WLAN, MAN, WAN, and the Internet. The network 130 may include wired and/or wireless communications according to one or more standards and/or via one or more transport mediums.

The communication over the network 130 may be performed in accordance with various communication protocols such as Transmission Control Protocol and Internet Protocol (TCP/IP), User Datagram Protocol (UDP), and IEEE communication protocols. In one example, the network 130 may include wireless communications according to Bluetooth specification sets or another standard or proprietary wireless communication protocol. In another example, the network 130 may also include communications over a cellular network, including, e.g., a GSM (Global System for Mobile Communications), CDMA (Code Division Multiple Access), EDGE (Enhanced Data for Global Evolution) network.

The system 100 is not confined to the components described herein and may include additional or other components, not shown for brevity, which are to be considered within the scope of the embodiments described herein.

The analytics server 110a may generate and display an electronic platform configured to use various computer models 111 (including artificial intelligence and/or machine learning models) to identify radiation parameters. More specifically, the platform may display one or more optimized (recommended, identified, selected) beam angles and/or a number of recommended beam angles (and the associated optimized beam angle). The number of recommended beam angles may be considered the field geometry settings for external beam radiotherapy. The electronic platform may include graphical user interface (GUI) displayed on each electronic data source 120, the end-user devices 140, the administrator computing device 150, and/or the medical device computer 162. An example of the electronic platform generated and hosted by the analytics server 110a may be a web-based application or a website configured to be displayed on different electronic devices, such as mobile devices, tablets, personal computer, and the like.

In a non-limiting example, a medical professional may input patient attributes, medical images, PTV and/or OAR information, radiotherapy treatment parameters (e.g., initial beam angles, field geometries, dose distributions, and the like) to electronic data source 120b. The medical professional operating devices 120b, 140c, and 162, may access the platform, review displayed revised beam angles (or sets of beam angles or other radiation therapy treatment parameters) generated from the machine learning model 111. Additionally or alternatively, the operations invoked by the analytics server 110a to optimize beam angle may be part of the operations in a sequence of operations to optimize a patient treatment plan (e.g., dose distribution among the patient's organs). The medical professional may use the medical professional device (e.g., medical professional device 140c) as both a device to display results predicted by the analytics server 110a and in some cases used as an electronic data source (e.g., electronic data source 120b) to train the machine learning models 111.

The analytics server 110a may recommend beam angles and other radiation parameters and/or treatment plan attributes used for proton radiation, photon radiation, and electron radiation. In particular, analytics server 110a may utilize the methods and systems described herein to automatically learn and recommend an improved (or optimized) beam angle. The analytics server 110a may display the beam angle and other radiation parameters and/or treatment plan attributes on an end-user device 140c, medical computing device 162, and/or a medical professional device 120b. The analytics server 110a may also use the beam angles and other radiation parameters and/or treatment plan attributes via one or more downstream applications. Further, the analytics server 110a may transmit the beam angles and other radiation parameters and/or treatment plan attributes to one or more other servers (e.g., clinic server 140b). Additionally, or alternatively, the analytics server 110a (or other server) may adjust the configuration of one of end-user devices 140 (e.g., the end-user device 140c) based on the optimized beam angle.

The analytics server 110a may host a website accessible to users operating any of the electronic devices described herein (e.g., end users, medical professionals), where the content presented via the various webpages may be controlled based upon each particular user's role or viewing permissions. The analytics server 110a may be any computing device comprising a processor and non-transitory machine-readable storage capable of executing the various tasks and processes described herein. The analytics server 110a may employ various processors such as central processing units (CPU) and graphics processing unit (GPU), among others. Non-limiting examples of such computing devices may include workstation computers, laptop computers, server computers, and the like. While the system 100 includes a single analytics server 110a, the analytics server 110a may include any number of computing devices operating in a distributed computing environment, such as a cloud environment.

The analytics server 110a may execute software applications configured to display the electronic platform (e.g., host a website), which may generate and serve various webpages to each electronic data source 120 and/or end-user devices 140. Different users may use the website to view and/or interact with the recommended (optimized) results. Different servers, such as server 120c and clinic server 140b may also use the recommended results in downstream processing. Additionally, or alternatively, the analytics server 110a may use the recommended beam angle to optimize one or more other radiation parameters and/or treatment plan attributes. For example, the analytics server 110a may use the recommended beam angle to optimize a dose distribution.

The analytics server 110a may be configured to require user authentication based upon a set of user authorization credentials (e.g., username, password, biometrics, cryptographic certificate, and the like). The analytics server 110a may access the system database 110b configured to store user credentials, which the analytics server 110a may be configured to reference in order to determine whether a set of entered credentials (purportedly authenticating the user) match an appropriate set of credentials that identify and authenticate the user.

The analytics server 110a may generate and host webpages based upon a particular user's role within the system 100. In such implementations, the user's role may be defined by data fields and input fields in user records stored in the system database 110b of the analytics server 110a. The analytics server 110a may authenticate the user and may identify the user's role by executing an access directory protocol (e.g., LDAP). The analytics server 110a may generate webpage content that is customized according to the user's role defined by the user record in the system database 110b.

The analytics server 110a may receive medical images from a user or retrieve such data from a data repository, analyze the data, and display the results on the electronic platform. For instance, in a non-limiting example, the analytics server 110a may query and retrieve medical images from the database 120d and combine the medical images with segment data received from a medical professional operating the medical professional device 120b. Additionally, or alternatively, the analytics server 110a may segment the medical image automatically or perform other pre-processing steps on the medical image captured from the medical device 140d.

The analytics server 110a may also perform other pre-processing steps on the medical image captured from the medical device 160. The analytics server 110a may execute various machine learning models 111 (stored within the system database of the clinic server 140b or the analytics server 110b) to analyze the retrieved data. The analytics server 110a may then display the results via the electronic platform on the administrator computing device 150, the medical professional device 120b, medical computing device 162 and/or the end-user devices 140.

The electronic data sources 120 may represent various electronic data sources that contain, retrieve, and/or input data associated with a patient's treatment plan including patient data and treatment data. For instance, the analytics server 110a may use the clinic computer 120a, medical professional device 120b, server 120c (associated with a physician and/or clinic), and database 120d (associated with the physician and/or the clinic) to retrieve/receive data associated with the patient's treatment plan.

End-user devices 140 may be any computing device comprising a processor and a non-transitory machine-readable storage medium capable of performing the various tasks and processes described herein. Non-limiting examples of an end-user device 140 may be a workstation computer, laptop computer, tablet computer, and server computer. In operation, various users may use end-user devices 140 to access the GUI operationally managed by the analytics server 110a. Specifically, the end-user devices 140 may include clinic computer 140a, clinic server 140b, and a medical device professional 140c. Even though referred to herein as “end user” devices, these devices may not always be operated by end users. For instance, the clinic server 140b may not be directly used by an end user. However, the results stored onto the clinic server 140b may be used to populate various GUIs accessed by an end user via the medical professional device 140c.

The administrator computing device 150 may represent a computing device operated by a system administrator. The administrator computing device 150 may be configured to display beam angles, radiation parameters and/or other radiation therapy treatment attributes generated by the analytics server 110a (e.g., various analytic metrics determined during training of one or more machine learning models and/or systems); monitor various models 111 utilized by the analytics server 110a, electronic data sources 120, and/or end-user devices 140; review feedback; and/or facilitate training or retraining (calibration) of the machine learning models 111 that are maintained by the analytics server 110a.

The medical device 160 may be a radiotherapy machine (e.g., a linear accelerator, particle accelerator (including circular accelerators), or a cobalt machine)) configured to implement a patient's radiotherapy treatment. The medical device 160 may also include an imaging device capable of emitting radiation such that the medical device 160 may perform imaging according to various methods to accurately image the internal structure of a patient. For instance, the medical device 160 may include a rotating system (e.g., a static or rotating multi-view system). A non-limiting example of a multi-view system may include a stereo systems (e.g., two systems may be arranged orthogonally). The medical device 160 may also be in communication with a medical device computer 162 that is configured to display various GUIs discussed herein. For instance, the analytics server 110a may display the results predicted by the machine learning model 111 onto the medical device computer 162.

In operation, a physician or other medical professional may access an application executing on the medical professional device 120b and input patient data and the patient's treatment data (e.g., patient information, patient diagnosis, radiation therapy radiation requirements and thresholds). The analytics server 110a then uses a patient identifier to query patient data (e.g., patient anatomy and/or medical images) from the electronic data sources 120. The analytics server may then identify a clinic associated with the patient (e.g., clinic performing the treatment) and retrieve one or more files associated with treatment templates and clinic rules. The analytics server 110a may then utilize the systems and methods described herein to generate optimized radiation parameters (e.g., beam angles or a treatment plan, in some configurations) and forward the optimized beam angles, other radiation parameters and/or treatment plan attributes to one or more downstream applications. The downstream application may, for example, employ additional machine learning models 111 to optimize a treatment plan based on the optimized beam angle. Additionally, or alternatively, the analytics server 110a may present for display the optimized beam angle results onto the medical professional device 120b, clinic computer 140a, medical computing device 162, and/or the medical device 260 (e.g., a display screen of the radiotherapy machine).

The analytics server 110a may be in communication (real-time or near real-time) with the computing device 162, end-user device 140 and/or electronic data sources 120, such that a server/computer hosting the medical device 160 can adjust the medical device 160 based on the beam angles, treatment attributes and/or radiation parameters revised by the analytics server 110a. For instance, the radiotherapy machine may adjust the gantry, beam blocking device (e.g. multi leaf collimator MLC), and couch based on optimized beam angles, where the optimized beam angle is an angle of the medical device 160 that emits radiation in a direction that maximizes radiation to PTVs and minimizes radiation to OARs. The analytics server 110a may transmit instructions to the radiotherapy machines indicating any number or type of radiation parameters and/or treatment attributes to facilitate such adjustments.

The analytics server 110a may store machine learning models 111 (e.g., neural networks, random forest, support vector machines, or other deep learning models), that are trained to predict (and improve or optimize) beam angles that optimize the treatment plan with respect to various clinical goals of patients at radiotherapy clinics. The analytics server 110a may train the machine learning models 111 using patient data and treatment data associated with patients who were previously treated. For instance, the analytics server 110a may receive patient data (e.g., physical attributes, diagnoses, 3D medical images) and treatment data recommended from one or more medical professionals (beam angles, does distributions, a number of beams for treatment, radiation type, radiation intensity) from any of the data sources 120 and clinical goals.

Machine learning models 111 may be stored in the system database 110b and may correspond to individual radiotherapy clinics or otherwise different sets of radiotherapy machines (e.g., radiotherapy machines that are located at individual radiotherapy clinics, are located in different geographical regions, treat specific types of diseases (e.g., different types of cancer), treat specific genders, etc.). For example, the machine learning model 111 may be associated with an identifier indicating the radiotherapy clinic, set of radiotherapy machines, or a specific disease for which it is configured to predict the probability of a reference point of a template image being at a location in 3D space.

A medical professional at a radiotherapy clinic may access an end-user device 140 located at the clinic or access an account associated with the clinic. The medical professional may provide an input at a user interface that causes the end user device 140 to transmit a request to access a machine learning model 111 that is associated with the clinic and/or the radiotherapy machines located within the clinic. The request may include an identifier associated with the machine learning model 111, the clinic, a treatment plan generated by the one or more medical professionals, and/or the set of radiotherapy machines that the analytics server 110a may use as a key in a look-up table to identify the machine learning model 111. The analytics server 110a may receive the request and, in some cases, after authenticating the user, identify the machine learning model 111 via the identifier. The analytics server 110a may transmit the identified machine learning model 111 to the end-user device 140 or send an alert indicating the end-user device is authorized to access the model(s) 111. Upon receipt or access to the machine learning model 111, the end user device 140 may perform the systems and methods described herein to train or retrain the machine learning model 111 to predict (and improve or optimize) beam angles.

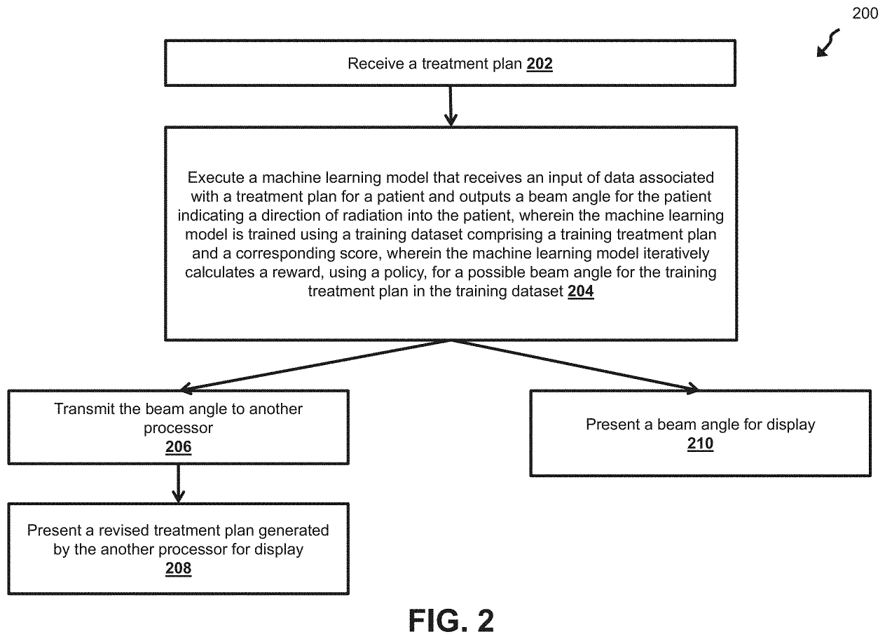

FIG. 2 illustrates a flow diagram of a process executed in a beam angle optimization system, according to an embodiment. The method 200 includes steps for determining a revised (optimized, improved, modified, identified, selected, and/or predicted) beam angle, set of beam angles or other radiation parameters and/or treatment attributes, according to an embodiment. As described herein, inputs and outputs may be described in the singular (e.g., ingest a medical image or output a revised beam angle). It should be appreciated that multiple inputs (e.g., medical images and corresponding PTV/OAR structures in the medical images) and multiple outputs (e.g., a set of beam angles, a set of radiation parameters, and the like) are considered. For example, treatment plans may consist of multiple treatment beans, where each treatment beam has a different angle. Accordingly, the method 200 may simultaneously improve all of the beam angles (and/or other radiation parameters) associated with a particular patient and a particular treatment plan. The method 200 may include steps 202-210. However, other embodiments may include additional or alternative steps, or may omit one or more steps altogether.

The method 200 is described as being executed by an analytics server, such as the analytics server described in FIG. 1. The analytics server may employ one or more CPUs and GPUs to perform one or more steps of method 200. The CPUs and/or GPUs may be performed in part by the analytics server and in part by one or more other servers and/or computing devices. The servers and/or computing devices employing the CPUs and GPUs may be local and/or remote (or some combination). For example, one or more virtual machines in a cloud may employ one or more CPUs and GPUs to perform one or more steps of method 200. A hybrid CPU and GPU implementation may improve the speed associated with training a machine learning model to select a beam angle. However, one or more steps of method 200 may be executed by any number of computing devices operating in the distributed computing system described in FIG. 1. For instance, one or more computing devices may locally perform part or all of the steps described in FIG. 2. Moreover, an “agent,” referring to the learner or the trainer (e.g., the analytics server training the machine learning model or the machine learning model itself), may perform one or more steps discussed herein.

In step 202, the analytics server may receive a treatment plan from one or sources (e.g., user devices such as physician device 120b, end-user devices 140 including the radiotherapy machine 140d, databases 110b, and electronic data sources 120 in FIG. 1). For instance, the analytics server may query one or more databases to identify medical data associated with the patient. The analytics server may query data associated with the patient's anatomy, such as physical data (e.g., height, weight, and/or body mass index) and/or other health-related data (e.g., blood pressure or other data relevant to the patient receiving radiation therapy treatment). The analytics server may also retrieve data associated with current and/or previous medical treatments received by the patient (e.g., data associated with the patient's previous surgeries).

The analytics server may analyze the data received and may generate additional queries accordingly. For instance, the analytics server may retrieve data associated with one or more medical (or other) devices needed for the patient. The analytics server may retrieve data indicating that the patient suffers from a respiratory medical condition. As a result, the analytics server may generate and transmit a query to one or more electronic data sources to identify whether the patient uses/needs a ventilator.

If necessary, the analytics server may also analyze the patient's medical data records to identify the needed patient attributes. For instance, the analytics server may query a database to identify the patient's body mass index (BMI). However, because many medical records are not digitalized, the analytics server may not receive the patient's BMI value using simple query techniques. As a result, the analytics server may retrieve the patient's electronic health data and may execute one or more analytical protocols (e.g., natural language processing) to identify the patient's body mass index. In another example, if the analytics server does not receive PTV and/or OAR data, then the analytics server may execute various image recognition protocols and segmentation to identify the PTV and/or OAR data in various medical images (e.g., planning images, simulation images, and diagnostic images). For example, the analytics server may automatically segment and/or pre-process various medical images using trained machine learning models. The machine learning models trained to segment medical images may generate contours on the medical images, segmenting the medical image and identifying one or more PTVs and/or OARs.

The treatment plan may be the plan determined by one or more medical professionals to treat a particular patient using radiation therapy. The treatment plan may include information such as a dose distribution, radiation parameters such as beam angles, field geometry settings, side effect prediction, organ and/or tumor segmentation, machine therapy attributes such as gantry position, couch position, beam blocking devices, treatment frequency, treatment timing, and treatment modalities, among others. The treatment plan may include a set of structures (e.g., PTVs and/or OARs) segmented from medical images and at least one defined beam angle.

The treatment plan may also include one or more medical images. The analytics server may transform or otherwise convert the medical image into a point cloud representation of structures (e.g., PTVs and/or OARs) in the medical images. An automatic image segmentation module may automatically segment PTVs and/or OARs in medical images. The medical images may be segmented, or otherwise analyzed to identify different contrasts in the medical image.

In some configurations, the medical images may be two-dimensional (2D). In other configurations, the medical images may be three-dimensional (3D). If the medical images are 2D, the analytics server may convert the 2D images into 3D images (e.g., using triangulation protocols, photogrammetry). Medical images may include CT scans, 4D CT scans, MRIs, and X-ray images, among others.

The treatment plan may also include one or more clinical goals. Clinical goals may include a dosimetric goodness function (e.g., a dosimentric quality or metric), a robustness measure, relative biological effects, and linear energy transfer metrics, among others. A medical professional may indicate one or more particular clinical goals for particular patients. For example, a medical professional may identify that a clinical goal associated with a younger patient is a robust treatment plan because the young patient may be more likely to move during treatment than an older patient. Additionally, or alternatively, clinical goals may include dose thresholds for PTVs and/or OARs. Clinical goals may also be associated with a particular clinic and/or particular radiotherapy machines. Additionally, or alternatively, a clinical goal may be predetermined (e.g., determined via an administrator computing device 150 in FIG. 1).

At step 204, the analytics server executes a machine learning model that receives an input of data associated with a treatment plan for a patient and outputs a set of beam angles for the patient indicating a set of directions direction of radiation into the patient. If the treatment plan contains an initial set of beam angles for the patient, the analytics server may output an improved (or optimized, recommended, selected, identified, revised) set of beam angles for the patient (e.g., a tensor indicating revised modifications to be applied to a beam angle).

By exploiting the fact that the core policy network of the machine learning model implicitly forecasts other radiation parameters and/or treatment plan attributes, machine learning models may be repurposed (e.g., retrained) to directly predict (or improve, select, recommend, and/or optimize) radiation parameters and/or treatment plan attributes such as dose distribution and/or dose intensity (e.g., dose prediction). Additionally, or alternatively, the analytics server may output one or more improved (or revised, optimized, selected, identified) other radiation treatment parameters such as a field geometry.

In some configurations, the treatment plan may not include an initial beam angle or other initial radiation parameter and/or treatment attribute. Accordingly, the analytics server will output a beam angle (e.g., not a revised beam angle because there is no beam angle to revise). That is, the analytics server may output a beam angle independent of receiving an initial beam angle in the treatment plan.

In other configurations, the analytics server may revise other treatment plan attributes (e.g., dose distribution) using other outputted treatment plan information (e.g., the revised beam angle). The analytics server may also output other treatment plan attributes. That is, the analytics server may output a treatment attribute independent of receiving various initial treatment attributes in the treatment plan.

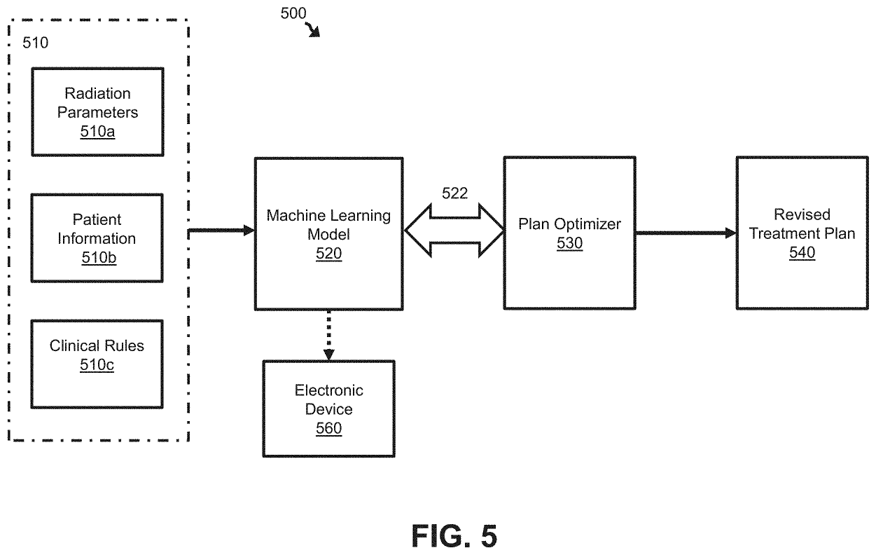

The executed machine learning model may be trained to revise (or optimize) beam angles for a patient using a training dataset including data ingested from previously performed treatments including treatment plans with corresponding medical images, radiation parameters (beam angles, field geometries), clinical goals, and a corresponding score representing a clinical quality, where the score is generated (e.g., by the analytics server and/or a second processor, such as plan optimizer 503 in FIG. 5) and the like. The analytics server and/or second processor may determine the score based on the clinical goals, dose distributions, beam angles, field geometries, of previously performed treatments. Additionally, or alternatively, a medical professional may score the clinical outcome of previously performed treatments.

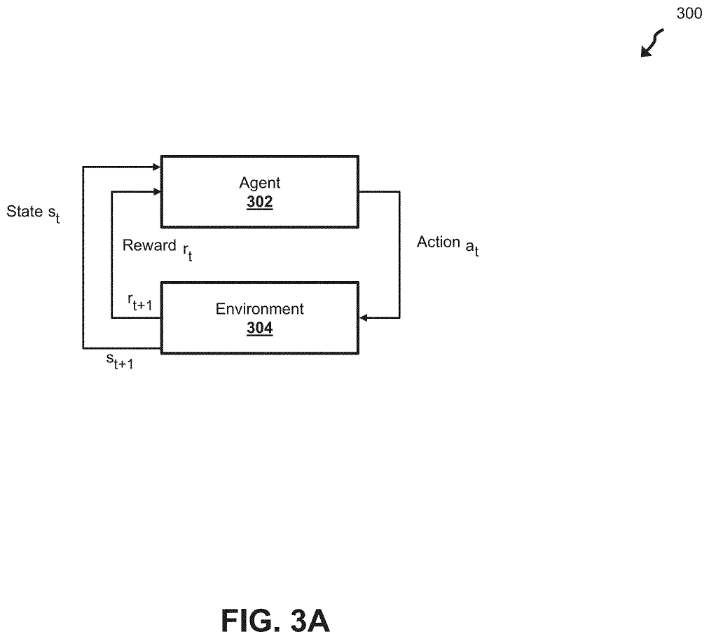

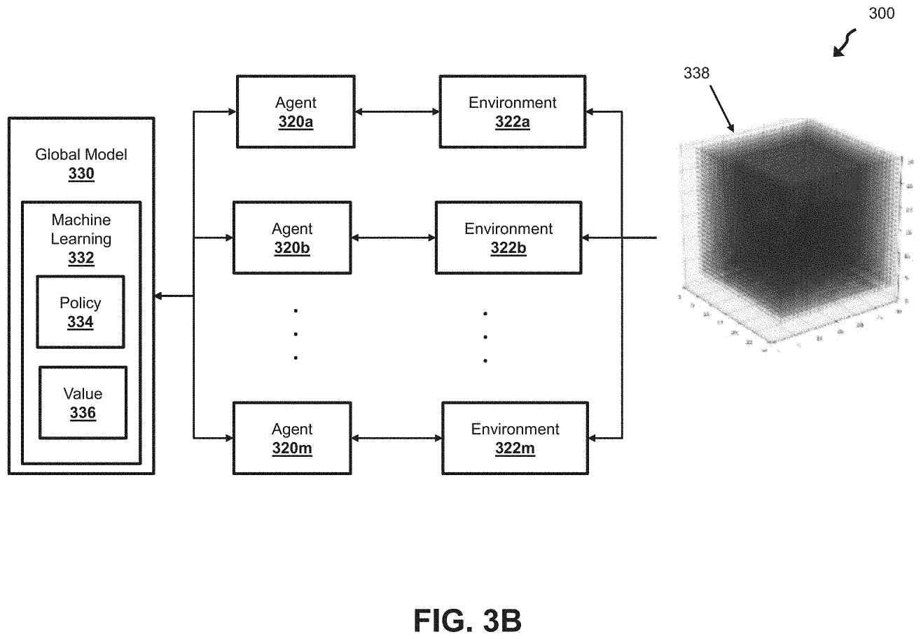

FIG. 3A illustrates a simplified reinforcement learning model 300, according to an embodiment. As used herein, the “agent” refers to the learner or the trainer (e.g., the analytics server training the machine learning model or the machine learning model itself). Agents use an initial treatment plan setup, attempt modifications (revisions, improvements, and/or optimizations) on parts of the treatment plan (e.g., radiation parameters, and in particular beam angles), learn whether the modifications were beneficial, and propose a set of modifications (e.g., recommended, improved, optimized beam angles) based on the trained reinforcement learning model 300.

In reinforcement learning, an agent 302 interacts with an environment 304. As discussed herein, an agent 302 refers to the learner or trainer. The environment 304 refers to encapsulated anatomical structures of the patient, as well as the positioning of the radiation fields in the initial treatment plan. At each time step t (e.g., each iteration), the agent 302 observes a state sₜ and selects an action from a set of actions. The possible set of actions may include increasing or decreasing a beam angle, not modifying a beam angle, and increasing or decreasing multiple beam angles simultaneously. The beam angles in the possible set of actions (e.g., action space) that may be associated with a particular position of the radiotherapy machine (e.g., a linear accelerator implemented in the medical device).

The possible set of actions (e.g., action space) may also be arbitrarily defined and depend on the solution space considerations. For example, the solution space may be discretized such that the possible angles for beams are at fixed intervals rather than on a continuous range. Accordingly, the action space may include actions such as “move in one direction,” “move in another direction,” or “don't modify y beam” where y is a specific beam. Additionally, or alternatively, the action space may include actions such as “move x steps in one direction,” “move x steps in the other direction,” or “don't modify y beam.”

The action space may include more complex schemes such as dual step-sizes for an explore/exploit approach. For example, the action space may include actions such as “move a small step in one direction,” “move a big step in one direction,” “move a small step in the other direction,” “move a big step in the other direction,” and “don't modify y beam.”

In other examples, the solution space may be continuous rather than discrete. For example, the action space may include actions such as “move x degrees” or “do not modify the beam position of y beam.” In the event a continuous solution space is implemented, the agents 302 may need to train for longer such that the agents 302 can determine, for example, in which direction there might be a better plan and how far in a certain direction a better plan may be.

Agents 302 may select an action based on the value of taking each action, where the value of selecting the action is defined as the expected reward received when taking that action from the possible set of actions. Agents 302 may select actions based on exploratory actions and exploitation actions. An exploratory action improves an agent's knowledge about an action by using the explored action in a sequence resulting in a reward calculation. An exploitation action is a “greedy” action that exploits the agent's 302 current action-value estimates. Using epsilon-greedy action selection, for example, the agent 302 balances exploratory actions and exploitation actions. The agent 302 may select an epsilon value and perform an exploitation action or an exploratory action based on the value of the epsilon and one or more exploitation and/or exploration thresholds. The agent 302 may randomly select an epsilon value and/or select an epsilon value from a predetermined distribution of epsilon values.

Agents 302 may also select an action using a policy 7C, where 7C maps states (and observations) to actions. The policy 7C gives the probability of taking a certain action when the agent is in a certain state.

In response to selecting an action (or multiple actions), the environment 304 may change, and there may be a new state sₜ₊₁. The agent 302 may receive feedback, indicating how the action affected the environment 304. In some configurations, the agent 302 determines the feedback. In other configurations, the analytics server may provide feedback. In yet other configurations, a second processor (e.g., plan optimizer 530 in FIG. 5) may provide feedback.

The agent 302 learns (e.g., reconfigures its policy π) by taking actions and analyzing the rewards received. A reward functions can include, for example, R(sₜ), R(sₜ, aₜ), and R(sₜ, aₜ, sₜ₊₁). In some configurations, the reward may be a dosimetric goodness function. For example, a reward function based on a dosimetric goodness function may include various quadratic terms representing considerations determined by a dosimetrist. Reward functions may also be based on other clinical goals including a robustness measure, relative biological effects, and linear energy transfer metrics, among others. Additionally or alternatively, a second processor (e.g., plan optimizer 530 in FIG. 5) may calculate rewards based on clinical goals, dosimetric goodness functions, robustness measures, relative biological effects, linear energy transfer metrics, among others, and provide the feedback to the agent 302.

Each iteration (or after multiple iterations and/or steps), the agent 302 selects a policy 7C (and an action) based on the current state sₜ and the agent 302 (or the machine learning model) calculates a reward. Each iteration, the agent 302 (or machine learning model) iteratively increases a summation of rewards.

One goal of reinforcement learning is to determine a policy 7C that maximizes the cumulative set of rewards, determined via the reward function. A core policy network evaluates the environment 304 and produces probabilistic distributions that the agent 302 (or the analytics server) uses to select how to modify the beam fields for a given possible beam angle.

The analytics server weighs policies based on the rewards determined at each step (or series of steps) such that certain policies (and actions) are encouraged and/or discouraged in response to the environment 304 being in a certain state. The policies are optimized by taking the gradient of an objective function (e.g., a reward function) to maximize a cumulative sum of rewards at each step, or after a predetermined number of steps (e.g., a delayed reward).

In some configurations, the rewards at each step may be compared (e.g., on an iterative basis) to a baseline. The baseline may be an expected performance (e.g., beam angle or other radiation parameter of the received treatment plan), or an average performance (e.g., an average beam angle over a series of steps). Evaluating a difference between the baseline and the reward is considered evaluating a value of advantage (or advantage value). The value of the advantage indicates how much better the reward is from the baseline (e.g., instead of an indication of which actions were rewarded and which actions were penalized).

In an example of training using reinforcement learning, a second processor (e.g., a plan optimizer 530 in FIG. 5) may determine a score using clinical goals (or other metrics). For example, the second processor may determine a score based on an initial treatment plan (e.g., a template treatment plan, a treatment plan proposed by a medical professional) by evaluating the initial treatment plan according to how well the clinical goals are met. The score assigned by the second processor may be used as the baseline by the agents 302 or the machine learning model. The machine learning model may compare a score associated with the treatment plan using the revised beam angle to the baseline score to evaluate whether the action selected by the agents 302 according to the policy should be punished or rewarded. Additionally or alternatively, the analytics server (including the agents 302 in the machine learning model) may determine and use a score based on the clinical goals.

Additionally or alternatively, the analytics server (including the agents 302 in the machine learning model or a different processor and/or different server) may measure the rewards (and/or associated beam angles) against metrics to assess the quality of the revised beam angles (sets of revised beam angles, or other radiation parameters and/or treatment information) and update the central policy network. For example, the baseline may be an expected reward. The analytics server (or a different process such as the plan optimizer 503 in FIG. 5) may evaluate the baseline by evaluating, for instance, a base treatment plan. The evaluation of the base treatment plan may include assigning a clinical quality score to the base treatment plan. The analytics server (or different processor such as the plan optimizer 503 in FIG. 5 and/or agents 302) may compare the evaluated base treatment plan to a revised treatment plan using the revised beam angles. Accordingly, the baseline may be expected clinical goals that can be correlated with the clinical quality of the revised treatment plan (or revised treatment parameter).

Additionally or alternatively, the analytics server (including the agents 302 in the machine learning model or a different processor and/or different server) may prioritize other metrics such as a measure of plan robustness, linear energy transfer (LET), and/or relative biological effects (RBE) rather than dosimetric quality and physical dose (or some combination).

The agents 302 trains themselves by choosing the action(s) based on policies that provide the highest cumulative set of rewards. The agents 302 of the machine learning model may continue training until a predetermined threshold has been satisfied. For instance, the analytics server may train the machine learning model until the advantage value is within a predetermined accuracy threshold. Additionally or alternatively, the analytics server may continue training the machine learning model until a predetermined number of steps (or series of steps called episodes, or iterations) have been reached.

The analytics server may determine weights to maximize the objective function (e.g., reward function) during training as shown in Equation 1 below.

$${▽}_{θ} J ( θ ) = \frac{1}{m} {\sum }_{i = 1}^{m} {\sum }_{t = 0}^{T} {▽}_{θ} log {π}_{θ} ( {a}_{t} \vert {s}_{t} ) ( ( Q ( {s}_{t} , {a}_{t} ) - {V}_{θ} ( {s}_{t} ) ) \\ log {π}_{θ} ( {a}_{t} \vert {s}_{t} ) is an agent term \\ Q ( {s}_{t} , {a}_{t} ) - {V}_{θ} ( {s}_{t} ) is a critic term \\ T is a number of steps in an episode \\ m is a number of episodes \qquad Equation ( 1 )$$

In Equation 1 above, agents 302 may approximate both the value function V_{θ}(sₜ) in the critic term and the policy function log π_{θ}(aₜ|sₜ) of the agent term. The agent term represents the probability of selecting a policy of taking an action aₜ given the state sₜ. The state-action value Q function in the critic term gives the expectation of the cumulative reward according to the current state sₜ after taking an action aₜ according to the current policy π. The value function in the critic term gives the expectation of the cumulative rewards at each step. The critic term may be approximated using Q actor-critic techniques, advantage actor-critic techniques, temporal difference (TD) actor-critic techniques, TD actor-critic techniques with a discount value, and natural actor-critic techniques, among others.

FIG. 3B illustrates asynchronous advantage actor critic reinforcement learning model 300, according to an embodiment. In model 300, the analytics server utilizes various asynchronous agents 320a, 320b, and 320m (collectively called agents 320) with each agent 320 having a corresponding environment 322a, 322b, and 322m (collectively called environment 322). The analytics server may employ a GPU to instantiate multiple learning agents in parallel. Environment 322 may be based on 3D point cloud 338, where the 3D point cloud 338 is based on a medical image. The 3D point cloud 338 may be a tensor-base point cloud representation of the structures (PTVs, OARs) in the patient's body. The environment 322 may be an encapsulation of the data used for each agent 320. The analytics server may convert information in the treatment plan into a serializable representation. For example, the environment 322 may include a copy of the 3D point cloud 338 and beam angle vector data (e.g., received from a treatment plan).

Each agent asynchronously performs actions and calculates rewards using a single machine learning model 322 (such as a deep neural network) in a global model 330. The analytics server may configure the machine learning model 322 to include sufficient layers to capture identifiers associated with various points in the 3D point cloud 338. That is, the layers of the machine learning model 332 may be used to determine relationships of the points in the 3D point cloud 338 and various PTV and/or OAR radiation sensitivities.

In some configurations, policies 334 and action values 336 are updated every step (or predetermined number of steps) based on the cumulative rewards determined by each agent 320. Action values 336 may be the values used in tandem with the policy 334 to act as a critic. Each agent may contribute to the global policy 334 and value 336 such that the total knowledge of the global model 330 increases and the global policy 334 learns how to best modify the treatment plans to achieve higher treatment performances. Each time the global model 330 is updated (e.g., after every step and/or predetermined number of steps), the analytics server propagates new weights back to agents 320 such that each agent shares common policies 334 and values 336.

The global model 330 allows each agent to have a more diversified training data and eliminates a need for synchronization of models associated with each agent 320. In other configurations, there may be models associated with each agent 320 and each agent may calculate a reward using a corresponding machine learning model.

In some configurations, the analytics server may update the global model 330 using agents 320 operating in other servers. That is, the analytics server may employ agents on other servers (e.g., via federated learning) to update the global model 330 and corresponding policy 334 and value 336.

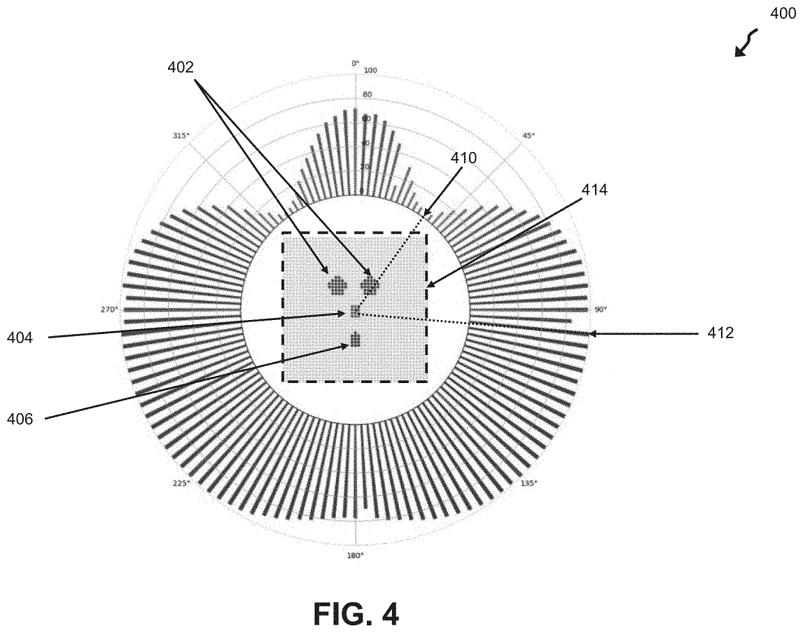

FIG. 4 illustrates a recommendation 400 of beam angles based on a treatment plan, according to an embodiment. For simplicity, a 2D image of a patient is used to generate a 2D point cloud 414. In other configurations, the image used to generate the 2D point cloud 414 may be synthetically defined from a geometric case. The 2D point cloud 414 includes points associated with the patient's eyes (e.g., points 402), points associated with the tumor (e.g., points 404), and points associated with the patient's spine (e.g., points 406). In response to receiving treatment plan information (e.g., including a medical image, clinical goals, PTV information, OAR information, and radiation dose information), the analytics server may determine one or more beam angles. Recommendation 400 illustrates a full pan of beam angles and corresponding rewards. A beam angle with a low reward is a beam angle that did not perform well with respect to one or more clinical goals. For example, a baseline beam angle may be associated with a higher reward than the beam angle determined by the analytics server.

In some configurations, the dosimetric goodness function describes tissue sensitivity, bone sensitivity, tumor sensitivity, and the like using, for example, a weighted sum of quadratic terms. The analytics server would have learned, during training, to avoid revising beam angles to go through points in the point cloud 414 associated with certain identifying information (e.g., OAR sensitivity information). Accordingly, beam angles indicating a direction of radiation into the patient through the patient's eyes (indicated by points 402) would not receive a high reward because points 402 may be associated with OAR sensitivity information. As shown, the analytics server determined that a 36 degree beam angle (indicated at 410) received a low reward, likely because the beam angle includes a direction of radiation through the patient's eyes (indicated by points 402) to irradiate the tumor (indicated by points 410). In contrast, the analytics server may determine that a 96 degree beam angle (indicated at 412) receives a high reward, likely because the beam angle does not include a direction of radiation through the patient's eyes (indicated by points 402) or spine (indicated by points 406).

Referring back to FIG. 2, in step 210, the analytics server may present for display a predetermined number of beam angles (e.g., a top five beam angles). In some configurations, the analytics server may present for display the full pan of beam angle evaluations (e.g., recommendation 400 in FIG. 4).

In step 206, the analytics server may transmit the beam angle (or set of beam angles) to another processor (or another computer). Referring to FIG. 5, a non-limiting visual example of a workflow utilizing the methods and systems described herein is illustrated. In this example 500, the analytics server provides beam angles to a plan optimizer 530 (e.g., a second processor) to provide a suggested (improved) treatment plan that is optimized for a patient. As discussed herein, the suggested treatment plan may be a revised (improved) version of the received treatment plan (e.g., step 202 in FIG. 2) based on beam angles determined by the machine learning model 520 (e.g., a first processor).

The analytics server may first receive a treatment plan for a particular patient 510. The treatment plan may include beam angles or other radiation parameters 510a (e.g., field geometries, dosage information), patient information 510b (e.g., medical images, PTVs, OARs), and clinical goals 510c (e.g., dosimetric goodness function, robustness metrics, biological effects of radiation, metrics based on linear energy transfer). The analytics server may train a machine-learning model 520 using previously performed radiation therapy treatments and corresponding scores representing a clinical quality. The trained machine-learning model 520 may then identify various beam angles to optimize (or improve, revise, select, predict, or identify) associated with the treatment plan for the particular patient 510.

The machine-learning model 520 may transmit the optimized (or improved, revised, selected, predicted or identified) beam angle for display to an electronic device 560. The electronic device may include a radiotherapy machine, a patient device, or an administrator device, among others.

The optimized beam angle determined via the machine learning model 520 may also be ingested by the plan optimizer 530. The plan optimizer 530 may be a treatment planning and/or monitoring software solution. The plan optimizer 530 may be executed by a second processor. For example, the plan optimizer 530 may be executed on a second computer. Additionally, or alternatively, the plan optimizer 530 may be executed on the processor executing machine learning model 520. The machine learning models 520 may be an ad-hoc external software capable of working in tandem with the plan optimizer 530.

The plan optimizer 530 may analyze various factors associated with the patient and the patient's treatment to generate and/or optimize (revise) a treatment plan for the patient (e.g., field geometry, treatment modality, dosage distribution, dosage prescription, radiation parameters). One of the factors considered by the plan optimizer 530 may be the beam angle outputted (identified, predicted, selected) by the machine-learning model 520.

While the plan optimizer 530 may consider beam angles as a factor, the plan optimizer 530 may weigh the beam angles differently than other factors considered to optimize (or generate) the patient's revised treatment plan. For instance, the revised treatment plan 540 determined by the plan optimizer 530 may not be dictated by the beam angles predicted by the machine-learning model 520. The plan optimizer 530 may utilize various cost function analysis protocols where the beam angles is evaluated in light of the other (sometimes more important) factors. In some cases, other factors may be prioritized over the beam angles.

The plan optimizer 530 may iteratively revise the patient's treatment plan by iteratively revising different attributes of the patient's treatment plan (e.g., field geometry, dose distribution). With each iteration, the plan optimizer 530 may transmit revised treatment plan data back to the machine-learning model 530 whereby the machine-learning model 530 can recalculate/re-predict new beam angles based on the revised treatment data generated by the plan optimizer (iteration 522). The plan optimizer 530 and the machine-learning model 520 may repeat the iteration 522 until the patient's treatment plan is optimized. When the plan optimizer 530 completes the patient's treatment plan, the plan optimizer 330 may transmit the revised (optimized, suggested) treatment plan 540 to one or more electronic devices where a user (e.g., medical professional) can review the revised treatment plan. For instance, the analytics server may display the revised treatment plan 540 on a computer of a clinic where a radiotherapy technician or a treating oncologist can review the treatment plan.

Generating a revised treatment plan may include evaluating a cost function. The objective of radiation therapy is to apply dosage satisfying a treatment threshold to a patient's PTV without applying dosage satisfying a harmful threshold to the patient's OAR. The plan optimizer 530 balances the amount of dosage received by OAR against the importance of the dosage to be applied to the PTV. In some cases, the plan optimizer 530 determine an acceptable amount of residual dosage at an OAR, such that PTV receives a proper amount of dosage. The plan optimizer 530 may minimize the dosage received by a patient's OAR (also referred to herein as the cost) and weigh the amount of dosage received against the dosage received by the patient's PTV.

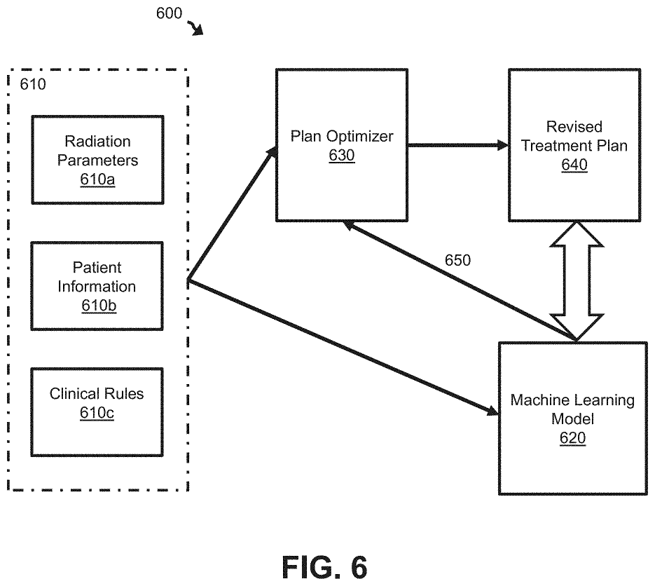

Referring now to FIG. 6, another non-limiting visual example of a workflow utilizing the methods and systems described herein is illustrated. In this example 600, the analytics server uses the methods discussed herein to evaluate a plan suggested (and improved) by a plan optimizer 630. In the depicted embodiment, the plan optimizer 630 and a machine-learning model 620 may work independently (as opposed to working together, as depicted in FIG. 5). The plan optimizer 630 may be executed by a second processor. For example, the plan optimizer 630 may be executed on a second computer. Additionally, or alternatively, the plan optimizer 630 may be executed on the processor executing the machine learning model 620.

The analytics server may first receive a treatment plan for a particular patient 610. As described above, the treatment plan 610 may include radiation parameters 610a (e.g., beam angles), patient information 610b (e.g., medical images), and clinical goals 610c (e.g., dosimetric goodness function, robustness metrics, biological effects of radiation, metrics based on linear energy transfer). The analytics server may train the machine-learning model 620 using previously performed radiation therapy treatments and a corresponding score representing a clinical quality, based on previously selected beam angles. The analytics server may then transmit the revised beam angles to the plan optimizer 630 where the plan optimizer 630 uses various analytical protocols and cost functions to generate a score to evaluate the revised beam angles. The plan optimizer may also optimize (or generate) a revised treatment plan for the patient 640 using the treatment plan 610.

The analytics server may then transmit the score, treatment plan 610 and/or the revised treatment plan 640 to the trained machine-learning model 620. The trained machine-learning model 620 may then use the score and the methods described herein to calculate revised (optimized, improved) beam angles for a treatment plan (e.g., treatment plan 610 and/or revised treatment plan 640).

Additionally or alternatively, the trained machine-learning mode 620 (or the analytics server) may transmit one or more revised radiation parameters values including beam angles back to the plan optimizer 630 (step 650). The plan optimizer 630 may then use the revised radiation parameters to recalculate a treatment plan for the patient, generate a score, and/or optimize a revised treatment plan 640 accordingly. Upon the plan optimizer 630 optimizing a revised treatment plan 640, the trained machine-learning model 620 may re-evaluate the revised treatment plan 640 using the methods described herein and a score produced by the plan optimizer 630, re-revising beam angles (or other radiation parameters). The plan optimizer 630 and the trained machine-learning model 620 may iteratively repeat this process where with each iteration the plan optimizer 630 updates the revised treatment plan 640 and the trained machine-learning model 620 re-evaluates the revised treatment plan 640 using the score. This iterative process may continue until the trained machine-learning model 620 determines that the revised treatment plan 640 is within tolerable thresholds (e.g., clinical goals, scores).

The various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of this disclosure or the claims.

Embodiments implemented in computer software may be implemented in software, firmware, middleware, microcode, hardware description languages, or any combination thereof. A code segment or machine-executable instructions may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

The actual software code or specialized control hardware used to implement these systems and methods is not limiting of the claimed features or this disclosure. Thus, the operation and behavior of the systems and methods were described without reference to the specific software code being understood that software and control hardware can be designed to implement the systems and methods based on the description herein.

When implemented in software, the functions may be stored as one or more instructions or code on a non-transitory computer-readable or processor-readable storage medium. The steps of a method or algorithm disclosed herein may be embodied in a processor-executable software module, which may reside on a computer-readable or processor-readable storage medium. A non-transitory computer-readable or processor-readable media includes both computer storage media and tangible storage media that facilitate transfer of a computer program from one place to another. A non-transitory processor-readable storage media may be any available media that may be accessed by a computer. By way of example, and not limitation, such non-transitory processor-readable media may comprise RAM, ROM, EEPROM, CD-ROM or other optical disk storage, magnetic disk storage or other magnetic storage devices, or any other tangible storage medium that may be used to store desired program code in the form of instructions or data structures and that may be accessed by a computer or processor. Disk and disc, as used herein, include compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and/or instructions on a non-transitory processor-readable medium and/or computer-readable medium, which may be incorporated into a computer program product.

The preceding description of the disclosed embodiments is provided to enable any person skilled in the art to make or use the embodiments described herein and variations thereof. Various modifications to these embodiments will be readily apparent to those skilled in the art, and the principles defined herein may be applied to other embodiments without departing from the spirit or scope of the subject matter disclosed herein. Thus, the present disclosure is not intended to be limited to the embodiments shown herein but is to be accorded the widest scope consistent with the following claims and the principles and novel features disclosed herein.

While various aspects and embodiments have been disclosed, other aspects and embodiments are contemplated. The various aspects and embodiments disclosed are for purposes of illustration and are not intended to be limiting, with the true scope and spirit being indicated by the following claims.

What we claim is:

1. A computer-implemented method of beam angle optimization comprising:
executing, by at least one processor, a machine learning model that receives an input of data associated with a treatment plan for a patient and outputs a beam angle for the patient indicating a direction of radiation into the patient,
wherein the machine learning model is trained using a training dataset comprising a training treatment plan and a corresponding score,
wherein the machine learning model iteratively calculates a reward, using a policy, for a possible beam angle for the training treatment plan in the training dataset, and
wherein the machine learning model iteratively increases a summation of rewards until the policy satisfies an accuracy threshold; and
transmitting, by the at least one processor, the beam angle to a second processor.

2. The computer-implemented method according to claim 1, wherein at least one of the treatment plan or the training treatment plan comprise at least one of a medical image, a clinical goal, a planning target volume, an organ at risk, a radiation type, a radiation dose, an initial beam angle, or a field geometry.

3. The computer-implemented method according to claim 2, wherein the medical image includes at least a structure of the planning target volume or a structure of the organ at risk.

4. The computer-implemented method according to claim 1, further comprising executing the machine learning model that receives the input of data associated with the treatment plan for the patient and outputs a dose distribution, wherein the machine learning model is trained using a training dataset comprising the training treatment plan and a corresponding score.

5. The computer-implemented method according to claim 1, further comprising presenting, by the processor, for display, the beam angle.

6. The computer-implemented method according to claim 1, wherein the machine learning model is trained using asynchronous advantage actor critic reinforcement learning.

7. The computer-implemented method according to claim 1, wherein the machine learning model is implemented using hybrid graphics processing units and central processing units.

8. The computer-implemented method according to claim 1, wherein the machine learning model is optimized with respect to one or more clinical goals received in the treatment plan, the clinical goals including at least one of a dosimetric quality, a robustness measure, metrics based on linear energy transfer, or relative biological effects.

9. The computer-implemented method according to claim 1, further comprising:
receiving, by the at least one processor from the second processor, a revised treatment plan, wherein the revised treatment plan is based on the beam angle;
executing, by the at least one processor, the machine learning model using the revised treatment plan for the patient and outputting a revised beam angle; and
transmitting, by the at least one processor, the revised beam angle to the second processor.

10. The computer-implemented method according to claim 1, wherein iteratively calculating the reward, using the policy, for the possible beam angle from the training treatment plan in the training dataset includes iteratively comparing the reward to a baseline.

11. A system comprising:
a server comprising a processor and a non-transitory computer-readable medium containing instructions that when executed by the processor causes the processor to perform operations comprising:
execute a machine learning model that receives an input of data associated with a treatment plan for a patient and outputs a beam angle for the patient indicating a direction of radiation into the patient, wherein the machine learning model is trained using a training dataset comprising a training treatment plan and a score, wherein the machine learning model iteratively calculates a reward, using a policy, for a possible beam angle for the training treatment plan in the training dataset, wherein the machine learning model iteratively increases a summation of rewards until the policy satisfies an accuracy threshold; and
transmit the beam angle to a second processor.

12. The system according to claim 11, wherein at least one of the treatment plan or the training treatment plan comprise at least one of a medical image, a clinical goal, a planning target volume, an organ at risk, a radiation type, a radiation dose, an initial beam angle, or a field geometry.

13. The system according to claim 12, wherein the medical image includes at least a structure of the planning target volume or a structure of the organ at risk.

14. The system according to claim 11, wherein the processor is further configured to execute the machine learning model that receives the input of data associated with the treatment plan for the patient and outputs a dose distribution, wherein the machine learning model is trained using a training dataset comprising the training treatment plan and a corresponding score.

15. The system according to claim 11, wherein the processor is further configured to present for display, the beam angle.

16. The system according to claim 11, wherein the machine learning model is trained using asynchronous advantage actor critic reinforcement learning.

17. The system according to claim 11, wherein the machine learning model is implemented using hybrid graphics processing units and central processing units.

18. The system according to claim 11, wherein the machine learning model is optimized with respect to one or more clinical goals received in the treatment plan, the clinical goals including at least one of a dosimetric quality, a robustness measure, metrics based on linear energy transfer, or relative biological effects.

19. The system according to claim 11, wherein the processor is further configured to:
receive, from the second processor, a revised treatment plan, wherein the revised treatment plan is based on the beam angle;
execute the machine learning model using the revised treatment plan for the patient and outputting a revised beam angle; and
transmit the revised beam angle to the second processor.

20. The system according to claim 11, wherein iteratively calculating the reward, using the policy, for the possible beam angle from the training treatment plan in the training dataset includes iteratively comparing the reward to a baseline.
